(19) United States
(12) Patent Application Publication
(10) Pub. No.: US 2026/0335327 A1
(43) Pub. Date: Oct. 29, 2026

(54) IDH MUTATIONS AS BIOMARKERS FOR ZOTIRACICLIB THERAPY

(71) Applicant: Cothera Bioscience, Inc., Grand Cayman (KY)

(72) Inventors: Xiang LI, Beijing (CN); Chun JIANG, Hillsborough, CA (US); Yiyou CHEN, San Jose, CA (US)

(21) Appl. No.: 19/101,501

(22) PCT Filed: Aug. 9, 2023
(86) PCT No.: PCT/US2023/071941

§ 371 Date: Feb. 5, 2025

(30) Foreign Application Priority Data
Aug. 10, 2022 (WO) PCT/CN2022/111327

(51) Int. Cl. A61K 31/529; A61K 45/06; A61P 35/00; C12Q 1/6813; C12Q 1/6869

(52) U.S. Cl. CPC A61K 31/529; A61K 45/06; A61P 35/00; C12Q 1/6813; C12Q 1/6869; C12Q 2600/106; C12Q 2600/156; G01N 2800/52; G01N 2800/7028

(57) ABSTRACT

Provided is the use of isocitrate dehydrogenase (IDH1 and IDH2) mutations for selecting and treating cancer patients for zotiraciclib (TG02) therapy, for example, in treating cancers such as glioma, medulloblastoma, chondrosarcoma, cholangiocarcinoma, AML, astrocytoma, and others, as a single agent of in conjunction with other chemotherapeutics

### CROSS-REFERENCE TO RELATED APPLICATIONS

This application claims priority to PCT/CN2022/111327, filed Aug. 10, 2022, which is incorporated by reference in its entirety.

### BACKGROUND

### Technical Field

Embodiments of the present disclosure relate to the use of isocitrate dehydrogenase (IDH1 and IDH2) mutations for selecting cancer patients for zotiraciclib (TG02) therapy, for example, in treating cancers such as glioma, medulloblastoma, chondrosarcoma, cholangiocarcinoma, AML, astrocytoma, and others.

### Description of the Related Art

Zotiraciclib (TG02) is a selective kinase inhibitor for the treatment of cancer (William et al., J. of Medicinal Chem. 55:169-196, 2012). It is an inhibitor of Cyclin Dependent Kinases (CDKs), Janus Kinase 2 (JAK2), and Fms-like Tyrosine Kinase-3 (FLT3), and is being evaluated in various clinical trials (see, for example, Wu et al., Clin Cancer Res. 27:3298-3306, 2021).

However, there is a need in the art to better predict the anti-cancer therapeutic efficacy of zotiraciclib, and thereby identify patients that will benefit most from treatment with this chemotherapeutic.

### BRIEF SUMMARY

Embodiments of the present disclosure include method for treating a cancer in a human subject in need thereof, wherein the cancer comprises an isocitrate dehydrogenase (IDH1 or IDH2) mutation, comprising administering to the subject a composition comprising zotiraciclib (TG02), or an analog, derivative, or pharmaceutically acceptable salt thereof, thereby treating the cancer comprising the IDH1 or IDH2 mutation. In specific embodiments, the cancer is glioma. Certain methods comprise (a) determining IDH1 or IDH2 mutation status in a tissue sample from the subject; and (b) administering to the subject a composition comprising zotiraciclib (TG02), or the analog, derivative, or pharmaceutically acceptable salt thereof, if the tissue sample comprises the IDH1 or IDH2 mutation.

In some embodiments, the IDH1 or IDH2 mutation is a gain-of-function mutation characterized by increased conversion of α-ketoglutarate (α-KG) into an oncometabolite D-2-hydroxyglutarate (D-2HG) relative to homozygous wild-type IDH1 or IDH2. In some embodiments, the IHD1 mutation is R132X, wherein X is selected from any amino acid other than R, optionally wherein the IHD1 mutation is R132C, R132G, R132H, R132L, or R132S. In some embodiments, the IHD2 mutation is R172X or R140X, wherein X is selected from any amino acid other R, optionally wherein the IHD2 mutation is R172G, R172K, R172M, R172S, R172T, or R140Q.

In some embodiments, step (a) comprises determining IDH1 or IDH2 mutation status in the tissue sample by DNA or RNA sequencing, in situ hybridization (ISH), fluorescence in situ hybridization (FISH), whole exome sequencing (WES), single nucleotide polymorphism (SNP) array, next generation sequencing (NGS), or comparative genome hybridization (CGH) on a human IDH1 or IDH2 protein or gene.

Certain embodiments include obtaining the tissue sample from the subject. In some embodiments, the tissue sample is a liquid biopsy optionally a blood sample, a surgical sample, or other biopsy sample obtained from the subject, optionally wherein the tissue sample is a cancer tissue sample.

In some embodiments, the cancer is selected from glioma (optionally low-grade or high-grade glioma), medulloblastoma, chondrosarcoma, cholangiocarcinoma, acute myeloid leukemia (AML), astrocytoma, sinonasal undifferentiated carcinoma (SNUC), angioimmunoblastic T cell lymphoma (AITL), and glioblastoma (optionally secondary glioblastoma).

Certain embodiments comprise administering to the subject an oral composition of zotiraciclib, or an analog, derivative, or pharmaceutically acceptable salt thereof. Some embodiments comprise administering the composition comprising zotiraciclib in combination with radiotherapy and/or one or more additional agents, optionally selected from chemotherapeutic agents, hormonal therapeutic agents, and/or kinase inhibitors. In some embodiments, the chemotherapeutic agent comprises an IDH1 or IDH2 inhibitor, optionally ivosidenib (AG-120), enasidenib, or AG-221.

Certain embodiments include the use of a diagnostic kit for treating a cancer in a human subject in need thereof with zotiraciclib (TG02), or an analog, derivative, or pharmaceutically acceptable salt thereof, wherein the cancer comprises an isocitrate dehydrogenase (IDH1 or IDH2) mutation, comprising means for determining isocitrate dehydrogenase (IDH1 or IDH2) mutation status in a tissue sample from the subject.

In some embodiments, the IDH1 or IDH2 mutation is a gain-of-function mutation characterized by increased conversion of α-ketoglutarate (α-KG) into an oncometabolite D-2-hydroxyglutarate (D-2HG) relative to homozygous wild-type IDH1 or IDH2. In some embodiments, the IHD1 mutation is R132X, wherein X is selected from any amino acid other than R, optionally wherein the IHD1 mutation is R132C, R132G, R132H, R132L, or R132S. In some embodiments, the IHD2 mutation is R172X or R140X, wherein X is selected from any amino acid other R, optionally wherein the IHD2 mutation is R172G, R172K, R172M, R172S, R172T, or R140Q.

In some embodiments, the means for determining IDH1 or IDH2 mutation status in a tissue sample comprise reagents for performing a diagnostic assay selected from one or more of DNA or RNA sequencing, in situ hybridization (ISH), fluorescence in situ hybridization (FISH), whole exome sequencing (WES), single nucleotide polymorphism (SNP) array, next generation sequencing (NGS), or comparative genome hybridization (CGH) on a human IDH1 or IDH2 protein or gene.

In some embodiments, the tissue sample is a liquid biopsy optionally a blood sample, a surgical sample, or other biopsy sample obtained from the subject, optionally a biopsy of prostate cancer tissue. In some embodiments, the cancer is selected from glioma (optionally low-grade or high-grade glioma), medulloblastoma, chondrosarcoma, cholangiocarcinoma, acute myeloid leukemia (AML), sinonasal undifferentiated carcinoma (SNUC), angioimmunoblastic T cell lymphoma (AITL), astrocytoma, and glioblastoma (optionally secondary glioblastoma). In some embodiments, the diagnostic/therapeutic kit comprises a composition comprising zotiraciclib, or an analog, derivative, or pharmaceutically acceptable salt thereof, optionally an oral composition of zotiraciclib. In some embodiments, the diagnostic/therapeutic kit comprises one or more additional agents, optionally selected from chemotherapeutic agents, hormonal therapeutic agents, and/or kinase inhibitors. In some embodiments, the chemotherapeutic agent comprises an IDH1 or IDH2 inhibitor, optionally ivosidenib (AG-120), enasidenib, or AG-221.

Also included are patient care kits, comprising: (a) means for determining isocitrate dehydrogenase (IDH1 or IDH2) mutation status in a tissue sample from a human subject with cancer; and (b) a composition comprising zotiraciclib (TG02), or an analog, derivative, or pharmaceutically acceptable salt thereof.

In some embodiments, the IDH1 or IDH2 mutation is a gain-of-function mutation characterized by increased conversion of α-ketoglutarate (α-KG) into an oncometabolite D-2-hydroxyglutarate (D-2HG) relative to homozygous wild-type IDH1 or IDH2. In some embodiments, the IHD1 mutation is R132X, wherein X is selected from any amino acid other than R, optionally wherein the IHD1 mutation is R132C, R132G, R132H, R132L, or R132S. In some embodiments, the IHD2 mutation is R172X or R140X, wherein X is selected from any amino acid other R, optionally wherein the IHD2 mutation is R172G, R172K, R172M, R172S, R172T, or R140Q.

In some embodiments, the means for determining IDH1 or IDH2 mutation status in a tissue sample comprise reagents for performing a diagnostic assay selected from one or more of DNA or RNA sequencing, in situ hybridization (ISH), fluorescence in situ hybridization (FISH), whole exome sequencing (WES), single nucleotide polymorphism (SNP) array, next generation sequencing (NGS), or comparative genome hybridization (CGH) on a human IDH1 or IDH2 protein or gene. In some embodiments, the tissue sample is a liquid biopsy optionally a blood sample, a surgical sample, or other biopsy sample obtained from the subject, optionally a biopsy of prostate cancer tissue.

In some embodiments, the cancer is selected from glioma (optionally low-grade or high-grade glioma), medulloblastoma, chondrosarcoma, cholangiocarcinoma, acute myeloid leukemia (AML), astrocytoma, sinonasal undifferentiated carcinoma (SNUC), angioimmunoblastic T cell lymphoma (AITL), and glioblastoma (optionally secondary glioblastoma).

In some embodiments, (b) comprises an oral composition of zotiraciclib, or an analog, derivative, or pharmaceutically acceptable salt thereof. In some embodiments, the patient care kit comprises one or more additional agents, optionally selected from chemotherapeutic agents, hormonal therapeutic agents, and/or kinase inhibitors. In some embodiments, the chemotherapeutic agent comprises an IDH1 or IDH2 inhibitor, optionally ivosidenib (AG-120), enasidenib, or AG-221.

Certain embodiments include a pharmaceutical composition for use in a method of treating a cancer in a human subject in need thereof, wherein the cancer comprises an isocitrate dehydrogenase (IDH1 or IDH2) mutation, comprising zotiraciclib (TG02), or an analog, derivative, or pharmaceutically acceptable salt thereof. In some embodiments, the IDH1 or IDH2 mutation is a gain-of-function mutation characterized by increased conversion of α-ketoglutarate (α-KG) into an oncometabolite D-2-hydroxyglutarate (D-2HG) relative to homozygous wild-type IDH1 or IDH2. In some embodiments, the IHD1 mutation is R132X, wherein X is selected from any amino acid other than R, optionally wherein the IHD1 mutation is R132C, R132G, R132H, R132L, or R132S. In some embodiments, the IHD2 mutation is R172X or R140X, wherein X is selected from any amino acid other R, optionally wherein the IHD2 mutation is R172G, R172K, R172M, R172S, R172T, or R140Q.

In some embodiments, the cancer is selected from glioma (optionally low-grade or high-grade glioma), medulloblastoma, chondrosarcoma, cholangiocarcinoma, acute myeloid leukemia (AML), astrocytoma, sinonasal undifferentiated carcinoma (SNUC), angioimmunoblastic T cell lymphoma (AITL), and glioblastoma (optionally secondary glioblastoma). Certain embodiments include the use of an oral composition of zotiraciclib, or an analog, derivative, or pharmaceutically acceptable salt thereof. Certain embodiments include the use of one or more additional agents, optionally selected from chemotherapeutic agents, hormonal therapeutic agents, and/or kinase inhibitors. In some embodiments, the chemotherapeutic agent comprises an IDH1 or IDH2 inhibitor, optionally ivosidenib (AG-120), enasidenib, or AG-221. In some embodiments, the method comprises: (a) determining IDH1 or IDH2 mutation status in a tissue sample from the subject; and (b) administering to the subject the composition comprising zotiraciclib (TG02), or the analog, derivative, or pharmaceutically acceptable salt thereof, if the tissue sample comprises the IDH1 or IDH2 mutation.

Also included is the use of a composition in the preparation of a medicament for treating a cancer in a human subject in need thereof, wherein the cancer comprises an isocitrate dehydrogenase (IDH1 or IDH2) mutation, comprising zotiraciclib (TG02), or an analog, derivative, or pharmaceutically acceptable salt thereof. In some embodiments, the IDH1 or IDH2 mutation is a gain-of-function mutation characterized by increased conversion of α-ketoglutarate (α-KG) into an oncometabolite D-2-hydroxyglutarate (D-2HG) relative to homozygous wild-type IDH1 or IDH2. In some embodiments, the IHD1 mutation is R132X, wherein X is selected from any amino acid other than R, optionally wherein the IHD1 mutation is R132C, R132G, R132H, R132L, or R132S. In some embodiments, the IHD2 mutation is R172X or R140X, wherein X is selected from any amino acid other R, optionally wherein the IHD2 mutation is R172G, R172K, R172M, R172S, R172T, or R140Q.

In some embodiments, the cancer is selected from glioma (optionally low-grade or high-grade glioma), medulloblastoma, chondrosarcoma, cholangiocarcinoma, acute myeloid leukemia (AML), astrocytoma, sinonasal undifferentiated carcinoma (SNUC), angioimmunoblastic T cell lymphoma (AITL), and glioblastoma (optionally secondary glioblastoma). Certain uses comprise an oral composition of zotiraciclib, or an analog, derivative, or pharmaceutically acceptable salt thereof. Particular uses comprise one or more additional agents, optionally selected from chemotherapeutic agents, hormonal therapeutic agents, and/or kinase inhibitors. In some embodiments, the chemotherapeutic agent comprises an IDH1 or IDH2 inhibitor, optionally ivosidenib (AG-120), enasidenib, or AG-221. Certain uses comprise the steps of (a) determining IDH1 or IDH2 mutation status in a tissue sample from the subject; and (b) administering to the subject the composition comprising zotiraciclib (TG02), or the analog, derivative, or pharmaceutically acceptable salt thereof, if the tissue sample comprises the IDH1 or IDH2 mutation.

### BRIEF DESCRIPTION OF THE FIGURES

FIG. 1 shows the 2-dimensional chemical structure of zotiraciclib.

FIGS. 2A-2B show that IDH1-mutant chondrosarcoma (HT-1080) and cholangiocarcinoma (RBE, Hcc-9810) cells are more sensitive to TG02 than IDH1-WT (Hucct-1) cells. Cells were treated with 0.2 μM TG02 for the indicated times. Apoptosis was determined by FACS assay

FIGS. 3A-3B shows that increased TG02 sensitivity can be conferred onto IDH1-WT (Hucct-1) cells by transfection of mutated IDH1. In FIG. 3A, Hucct-1 cells were transfected with IDH1 R132H plasmid and treated with 0.1 μM TG02 for 72 h. Apoptosis was determined by FACS assay. In FIG. 3B, Hucct-1 cells were transfected with IDH1 R132H plasmid, and the levels of D-2-hydroxyglutarate (D-2HG) were measured in Hucct-1 WT, Hucct-1 transfected with IDH1 R132H plasmid, and RBE (cholangiocarcinoma cell line with IDH1 R132S mutation) cells. D-2HG is a metabolic biomarker of gain-of-function mutation(s) in the IDH1 and/or IDH2 genes.

FIG. 4 shows that TG02-induced apoptosis of mutant IDH1 (Hccc-9810) cells can be rescued by addition of an excess of exogenous substrate α-ketoglutarate (α-KG). Mutant IDH1/2 converts α-KG into the oncometabolite D-2HG, which then inhibits a class of α-KG-dependent enzymes involved in epigenetic regulation. Cells were pretreated with 24 h with α-KG for 24 h, then treated with TG02 for 72 h. Apoptosis was determined by FACS assay.

FIGS. 5A-5B show that TG02-induced DNA damage in mutant IDH1 (HT-1080) cells can be rescued by addition of exogenous α-KG or AG-120. AG-120 is an inhibitor of mutant IDH1. In FIG. 5A, cells were treated with indicated drugs for 48 h, and DNA damage was determined by the neutral comet assay. FIG. 5B shows statistical analyses of the data in 5A (Left panels: Tail moment. Right panels: positive percent of cells with DNA damage).

FIG. 6 shows that TG02-induced DNA damage was rescued only in mutant IDH1 cells (Hccc-9810) by addition of exogenous AG-120. Cells were treated with indicated drugs for 48 h. pH2AX, the marker of DNA damage, was assayed by WB

FIGS. 7A-7B show that cells with IDH1 mutation are more sensitive to DNA damage induced by TG02. In FIG. 7A, IDH1-mutant Hucct1 cells (Hucct1^{R132H/+}) were generated by transduction with a lentivirus-expressing IDH1 R132H. Hucct1, Hucct1^{R132H/+}, and HT-1080 cells were treated with 0.2 μM TG02 or 0.1% DMSO for 48 h, and analyzed by comet assays. Quantification of Tail moment in neutral comet assay is presented. ns: no significant differences; P<0.001, ****: P<0.0001, compared with NC; #: P<0.05, compared with Hucct1 cells treated with TG02. In FIG. 7B, Hucct1 and Hucct1^{R132H/+} cells were treated with 2 μM AG120, 0.2 μM TG02 or 0.01 μM AZD4573 (CDK9 inhibitor) as indicated. Shown is Western blot analysis of phosphorylated γH2AX (a marker of DSBs) after being treated for 16 h. β-actin was used as a loading control.

FIGS. 8A-8C show that TG02 combined with AG120 induced cell apoptosis in cells with mutant IDH1. Mutant IDH1 cells HT-1080 (8A), RBE (8B), and Hccc-9810 (8C) were treated with TG02 (0.1 μM) and AG120 (2 μM) as indicated for 48 h, and apoptosis was measured by Annexin V/PI staining and analysis by flow cytometry.

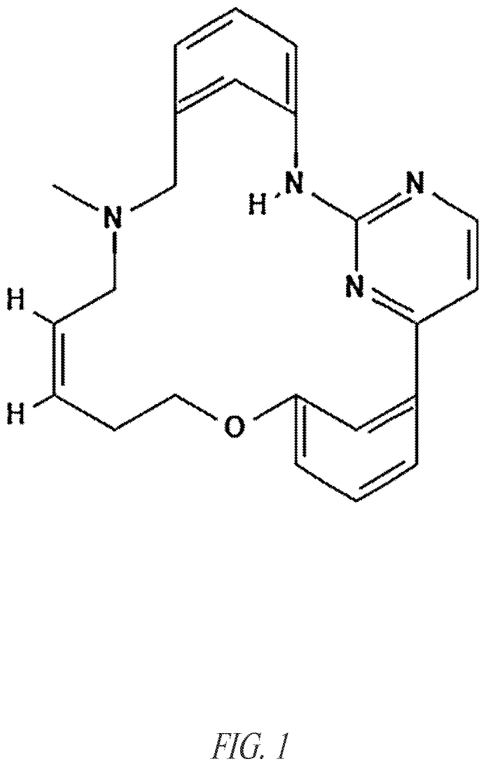

### DETAILED DESCRIPTION

Embodiments of the present disclosure relate to the surprising discovery that cancers comprising an isocitrate dehydrogenase (IDH1 and IDH2) mutation show significantly higher sensitivity to zotiraciclib (TG02) therapy. Thus, IDH1/2 mutation status and its associated gain-of-function phenotype can be used as biomarkers or companion diagnostics to select patients for optimized zotiraciclib cancer therapies.

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

For the purposes of the present disclosure, the following terms are defined below.

The articles “a” and “an” are used herein to refer to one or to more than one (i.e., to at least one) of the grammatical object of the article. By way of example, “an element” means one element or more than one element.

An “antagonist” or “inhibitor” refers to biological structure or chemical agent (e.g., compound) that interferes with or otherwise reduces the physiological action of another molecule, such as a protein. In some instances, the antagonist or inhibitor specifically binds to the other molecule and/or a functional ligand of the other molecule. In some instances, the antagonist or inhibitor down-regulates the expression of the other molecule. Included are full and partial antagonists.

An “agonist” or “activator” refers to biological structure or chemical agent that increases or enhances the physiological action of another agent or molecule. In some instances, the agonist specifically binds to the other agent or molecule. Included are full and partial agonists.

By “about” is meant a quantity, level, value, number, frequency, percentage, dimension, size, amount, weight or length that varies by as much as 20, 15, 10, 9, 8, 7, 6, 5, 4, 3, 2, or 1% to a reference quantity, level, value, number, frequency, percentage, dimension, size, amount, weight, or length.

The term “binding” refers to a direct association between two molecules, due to, for example, covalent, electrostatic, hydrophobic, and ionic and/or hydrogen-bond interactions, including interactions such as salt bridges and water bridges.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “half maximal effective concentration” or “EC₅₀” refers to the concentration of an agent (e.g., compound) as described herein at which it induces a response halfway between the baseline and maximum after some specified exposure time; the EC₅₀ of a graded dose response curve therefore represents the concentration of an agent at which 50% of its maximal effect is observed. EC₅₀ also represents the plasma concentration required for obtaining 50% of a maximum effect in vivo. Similarly, the “EC₉₀” refers to the concentration of an agent or composition at which 90% of its maximal effect is observed. The “EC₉₀” can be calculated from the “EC₅₀” and the Hill slope, or it can be determined from the data directly, using routine knowledge in the art. In some embodiments, the EC₅₀ of an agent is less than about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, 100, 200 or 500 nM. In some embodiments, an agent will have an EC₅₀ value of about 1 nM or less.

The “half maximal inhibitory concentration” (or “IC₅₀”) is a measure of the potency of an agent in inhibiting a specific biological or biochemical function. This quantitative measure indicates how much of a particular agent (inhibitor) is needed to inhibit a given biological process (or component of a process, i.e. an enzyme, cell, cell receptor or microorganism) by half. The values are typically expressed as molar concentration. The concentration is commonly used as a measure of antagonist drug potency in pharmacological research. In some instances, IC₅₀ represents the concentration of an agent that is required for 50% inhibition in vitro. The IC₅₀ of an agent can be determined by constructing a dose-response curve and examining the effect of different concentrations of the agent on the desired activity, for example, inhibition of tumor cell proliferation, tumor-cell killing.

The “half-life” of an agent refers to the time it takes for the agent to lose half of its pharmacologic, physiologic, or other activity, relative to such activity at the time of administration into the serum or tissue of an organism, or relative to any other defined time-point. “Half-life” can also refer to the time it takes for the amount or concentration of an agent to be reduced by half of a starting amount administered into the serum or tissue of an organism, relative to such amount or concentration at the time of administration into the serum or tissue of an organism, or relative to any other defined time-point. The half-life can be measured in serum and/or any one or more selected tissues.

The terms “modulating” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an amount that is about or at least about 1.1, 1.2, 1.5, 2, 3, 4, 5, 6, 7, 8, 9, 10, 15, 20, 30, 40, 50, 60, 70, 80, 90, 100, 200, 300, 400, 500, 600, 700, 800, 900, 1000, 2000, 3000, 4000, 5000-fold or more of the amount produced by no composition or a control composition (e.g., the absence of agent or a different agent). An “increased,” “stimulated” or “enhanced” amount may also include an amount that is about or at least about 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, 2000%, 3000%, 4000%, 5000% or more of the amount produced by no composition or a control composition. A “decreased” or “reduced” amount is typically a “statistically significant” amount, and may include an amount that is about or at least about 1.1, 1.2, 1.5, 2, 3, 4, 5, 6, 7, 8, 9, 10, 15, 20, 30, 40, 50, 60, 70, 80, 90, 100, 200, 300, 400, 500, 600, 700, 800, 900, 1000, 2000, 3000, 4000, or 5000-fold less of the amount produced by no composition or a control composition. A “decreased” or “reduced” amount may also include a 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100%, 200%, 300%, 400%, 500%, 600%, 700%, 800%, 900%, 1000%, 2000%, 3000%, 4000%, or 5000% less of the amount produced by no composition or a control composition. Examples of comparisons and “statistically significant” amounts are described herein.

“Prodrug” is meant to indicate an agent (e.g., compound) that may be converted under physiological conditions or by solvolysis to a biologically active compound described herein. Thus, the term “prodrug” refers to a metabolic precursor of a compound that is pharmaceutically acceptable. A prodrug may be inactive when administered to a subject in need thereof, but is converted in vivo to an active compound. Prodrugs may be rapidly transformed in vivo to yield the parent compound, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., A.C.S. Symposium Series, Vol. 14, and in Bioreversible Carriers in Drug Design, Ed. Edward B. Roche, American Pharmaceutical Association and Pergamon Press, 1987. Examples of prodrugs include, but are not limited to, acetate, formate, and benzoate derivatives of alcohol or amide derivatives of amine functional groups in the compounds of the disclosure and the like.

The term “prodrug” is also meant to include any covalently bonded carriers, which release the active compound in vivo when such prodrug is administered to a subject. Prodrugs of a compound may be prepared by modifying functional groups present in the compound in such a way that the modifications are cleaved, either in routine manipulation or in vivo, to the parent compound. Prodrugs include compounds where a hydroxy, amino, or mercapto group is bonded to any group that, when the prodrug of the compound is administered to a subject, cleaves to form a free hydroxy, free amino, or free mercapto group, respectively.

“Pharmaceutically-acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier, for example, which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

As used herein, the term “solvate” refers to an aggregate that comprises one or more molecules of an agent (e.g., compound) described herein with one or more molecules of solvent. The solvent may be water, in which case the solvate may be a hydrate. Alternatively, the solvent may be a biologically-inert organic solvent. Thus, the compounds described herein may exist as a hydrate, including a monohydrate, dihydrate, hemihydrate, sesquihydrate, trihydrate, tetrahydrate and the like, as well as the corresponding solvated forms. The compound of the disclosure may be true solvates, while in other cases, the compound may merely retain adventitious water or be a mixture of water plus some adventitious solvent.

A “pharmaceutical composition” refers to a formulation of a zotiraciclib (TG02) compound described herein and a medium generally accepted in the art for the delivery of the biologically active compound to mammals, e.g., humans. Such a medium includes all pharmaceutically acceptable carriers, diluents, and excipients.

The zotiraciclib compounds described herein, or their pharmaceutically-acceptable salts, may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

In certain embodiments, the “purity” of any given agent in a composition may be defined. For instance, certain compositions may comprise an agent that is at least 80%, 85%, 90%, 91%, 92%, 93%, 94%, 95%, 96%, 97%, 98%, 99%, or 100% pure on a weight-weight basis, including all decimals and ranges in between, as measured, for example and by no means limiting, by high performance liquid chromatography (HPLC), a well-known form of column chromatography used frequently in biochemistry and analytical chemistry to separate, identify, and quantify agents or compounds.

The term “solubility” refers to the property of an agent provided herein to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, pH 7.4, pH 7.6, pH 7.8, or pH 8.0 (e.g., about pH 5-8). In certain embodiments, solubility is measured in water or a physiological buffer such as PBS or NaCl (with or without NaPO4). In specific embodiments, solubility is measured at relatively lower pH (e.g., pH 6.0) and relatively higher salt (e.g., 500 mM NaCl and 10 mM NaPO4). In certain embodiments, solubility is measured in a biological fluid (solvent) such as blood or serum. In certain embodiments, the temperature can be about room temperature (e.g., about 20, 21, 22, 23, 24, 25 °C) or about body temperature (37 °C). In certain embodiments, an agent has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90 or 100 mg/ml at room temperature or at 37 °C.

“Stable compound” and “stable structure” are meant to indicate a compound that is sufficiently robust to survive isolation to a useful degree of purity from a reaction mixture, and formulation into a therapeutic agent.

The term “polynucleotide” and “nucleic acid” includes mRNA, RNA, CRNA, cDNA, and DNA including genomic DNA. The term typically refers to polymeric form of nucleotides of at least 10 bases in length, either ribonucleotides or deoxynucleotides or a modified form of either type of nucleotide. The term includes single and double stranded forms of DNA.

A “gene” refers to a hereditary unit consisting of a sequence of DNA that occupies a specific location on a chromosome and codes for a functional molecule or protein. The structure of a gene consists of many elements of which the actual protein coding sequence is often only a small part. These elements include DNA regions that are not transcribed as well as untranslated regions of the RNA. Additionally, genes can have expression-altering regulatory regions that lie many kilobases upstream or downstream of the coding sequence. The information in a gene can also be represented by (or found in) a sequence of RNA or encoded protein.

A “subject” or a “subject in need thereof” includes a mammalian subject such as a human subject.

By “statistically significant” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Substantially” or “essentially” means nearly totally or completely, for instance, 95%, 96%, 97%, 98%, 99% or greater of some given reference quantity, level, value, number, frequency, percentage, dimension, size, amount, weight, length, or other.

A “stereoisomer” refers to a compound made up of the same atoms bonded by the same bonds but having different three-dimensional structures, which are not interchangeable. The present disclosure includes various stereoisomers and mixtures thereof and includes “enantiomers”, which refers to two stereoisomers whose molecules are non-superimposable mirror images of one another.

A “tautomer” refers to a proton shift from one atom of a molecule to another atom of the same molecule. The present disclosure includes tautomers of any said compounds.

“Therapeutic response” refers to improvement of symptoms (whether or not sustained) based on the administration of the therapeutic response.

As used herein, the terms “therapeutically effective amount”, “therapeutic dose,” “prophylactically effective amount,” or “diagnostically effective amount” is the amount of an agent needed to elicit the desired biological response following administration.

As used herein, “treatment” of a subject (e.g. a mammal, such as a human) or a cell is any type of intervention used in an attempt to alter the natural course of the subject or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The term “wild-type” refers to a gene or gene product (e.g., a polypeptide) that is most frequently observed in a population and is thus arbitrarily designed the “normal” or “wild-type” form of the gene.

Each embodiment in this specification is to be applied to every other embodiment unless expressly stated otherwise.

Certain embodiments include methods for treating a cancer in a human subject in need thereof, wherein the cancer comprises an isocitrate dehydrogenase (IDH1 or IDH2) mutation, comprising administering to the subject a pharmaceutical composition comprising zotiraciclib (TG02), or an analog, derivative, or pharmaceutically acceptable salt thereof, thereby treating the cancer comprising the IDH1 or IDH2 mutation.

Some embodiments for treating an IDH1 or IDH2 mutated cancer include:
(a) determining IDH1 or IDH2 mutation status in a tissue sample from the subject; and
(b) administering to the subject a pharmaceutical composition comprising zotiraciclib (TG02), or the analog, derivative, or pharmaceutically acceptable salt thereof, if the tissue sample comprises the IDH1 or IDH2 mutation.

Also included are methods for predicting therapeutic response to zotiraciclib (TG02), or an analog, derivative, or pharmaceutically acceptable salt thereof, in a human subject with cancer, comprising:
(a) determining IDH1 or IDH2 mutation status in a tissue sample from the subject; and
(b) (i) characterizing the subject as responsive to zotiraciclib (TG02) therapy if the tissue sample comprises the IDH1 or IDH2 mutation; or
   (ii) characterizing the subject as non-responsive to zotiraciclib (TG02) therapy if the tissue sample does not comprise the IDH1 or IDH2 mutation, for example, if the sample comprises homozygous wild-type IDH1 and IDH2,
thereby predicting therapeutic response to zotiraciclib (TG02) in the subject with cancer.

Some embodiments include administering zotiraciclib to the subject if the subject is characterized as responsive to zotiraciclib therapy. Some instances include administering to the subject a chemotherapeutic agent excluding zotiraciclib if the subject is characterized as non-responsive to zotiraciclib therapy.

“Zotiraciclib” or “TG02” refers to the small molecule having the IUPAC Name: (16E)-14-methyl-20-oxa-5,7,14,27-tetraazatetracyclo[19.3.1.1^{2,6}. 1^{8,12}]heptacosa-1(25),2(27),3,5,8,10,12(26),16,21,23-decaene, the PubChem CID: 16739650, and CAS Number: 1204918-72-8, and includes pharmaceutically-acceptable salts and acids thereof. Also included are biologically-active or equivalent analogs and/or derivatives of zotiraciclib, including prodrugs and pharmaceutically-acceptable salts thereof.

The term “isocitrate dehydrogenase” or “IDH” refers to enzymes (and encoding IDH genes) that catalyze the oxidative decarboxylation of isocitrate, producing α-ketoglutarate and CO2. The two-step process involves oxidation of isocitrate (a secondary alcohol) to oxalosuccinate (a ketone), followed by the decarboxylation of the carboxyl group beta to the ketone, forming α-ketoglutarate. In humans, IDH exists in three isoforms: IDH1 (Uniprot: O75874), IDH2 (Uniprot: P48735), and IDH3 (Uniprot: P50213, O43837, and P51553). The IDH3 isoform is composed of three subunits and catalyzes the third step of the citric acid cycle while converting NAD+ to NADH in the mitochondria. The IDH1 and IDH2 isoforms catalyze the same reaction outside the context of the citric acid cycle and use NADP+ as a cofactor instead of NAD+.

As noted above, certain embodiments comprise administering zotiraciclib to the subject if the tissue sample comprises an IDH1 or IDH2 mutation relative to wild-type IDH1 or IDH2. Exemplary IDH1 and IDH2 mutations in cancer are described in the art (see, for example, Pirozzi and Yan, Nature Reviews Clinical Oncology. 18:645-661, 2021; and Persico et al., Cancers (Basel). 14(5); 1125, 2022, doi: 10.3390/cancers14051125). In certain embodiments, the IHD1 mutation is R132X, wherein X is selected from any amino acid other than R (arginine). In specific embodiments, the IHD1 mutation is R132C, R132G, R132H, R132L, or R132S. In some embodiments, the IHD2 mutation is R172X or R140X, wherein X is selected from any amino acid other R (arginine). In specific embodiments, the IHD2 mutation is R172G, R172K, R172M, R172S, R172T, or R140Q. In certain embodiments, the IDH1 or IDH2 mutation is a “gain-of-function” mutation characterized by increased conversion of α-ketoglutarate (α-KG) into the oncometabolite D-2-hydroxyglutarate (D-2HG) relative to homozygous wild-type IDH1 or IDH2 (see, for example, Chowdhury et al., EMBO Rep. 12 (5): 463-9, 2011). Certain embodiments thus include determining the levels, presence, or absence of the D-2HG oncometabolite in the tissue sample, for instance, and administering zotiraciclib to the subject if levels of the D-2HG oncometabolite are present or increased in the tissue sample relative to a reference or standard (e.g., D-2HG levels in a homozygous wild-type IDH1 or IDH2 tissue sample or cell).

IDH1 or IDH2 mutation status in a tissue sample can be determined by any variety of methods. For instance, in some embodiments, IDH1 or IDH2 mutation status is determined directly, for example, by DNA or RNA sequencing, in situ hybridization (ISH), fluorescence in situ hybridization (FISH), whole exome sequencing (WES), single nucleotide polymorphism (SNP) array, next generation sequencing (NGS), or comparative genome hybridization (CGH) on a human IDH1 or IDH2 protein or gene. CGH refers to a molecular cytogenetic method for analyzing copy number variations (CNVs) relative to ploidy level in the DNA of a test sample compared to a reference sample, without the need for culturing cells. This technique allows quick and efficient comparisons between two genomic DNA samples arising from two sources, which are most often closely related, because it is suspected that they contain differences in terms of either gains or losses of either whole chromosomes or subchromosomal regions (a portion of a whole chromosome). The technique was originally developed for the evaluation of the differences between the chromosomal complements of solid tumor and normal tissue (see, e.g., Kallioniemi et al., Science. 258 (5083): 818-821, 1992). The use of DNA microarrays in conjunction with CGH techniques has led to the development of a more specific form of array CGH (aCGH), allowing for a locus-by-locus measure of CNV with increased resolution as low as 100 kilobases (see, e.g., Pinkel, Annu Rev Genom Hum Genet. 6:331-354, 2005). In situ hybridization (ISH) and fluorescent in situ hybridization (FISH) refer to a type of hybridization that uses a labeled complementary DNA, RNA or modified nucleic acids strand (i.e., probe) to localize a specific DNA or RNA sequence in a portion or section of tissue (in situ) (see, e.g., Parra & Windle, Nature Genetics. 5:17-21, 1993; and Gall & Pardue, PNAS USA. 63:378-383, 1969). Thus, the step of determining IDH1 or IDH2 status, for example, to identify IDH1 or IDH2 mutations of interest (or their absence), can be performed according to routine techniques in the art. In some instances, the methods and kits described herein employ any one or more of the foregoing techniques and/or comprise reagents for performing the same.

In some embodiments, IDH1 or IDH2 mutation status is determined indirectly, for example, by determining D-2HG levels in a tissue sample. D-2HG levels can be determined or measured according to a variety of techniques in the art, including biochemical detection (e.g., colorimetric assays), biosensors, gas or liquid chromatography-mass spectrometry (GC- or LC-MS), matrix-assisted laser desorption ionization-time of flight mass spectrometry (MALDI-TOF), and others (see, for example, Xiao et al., Nat Commun 12:7108, 2021; the D-2HG assay in Example 1; Longuespée et al., Acta Neuropathol Commun. 6:21, 2018). Thus, the step of determining D-2HG levels, for example, to indirectly determine IDH1 or IDH2 mutation status, can be performed according to routine techniques in the art. In some instances, the methods and kits described herein employ any one or more of the foregoing techniques and/or comprise reagents for performing the same.

Examples of a “reference” include a value, amount, sequence, or other characteristic obtained from a database, for example, a homozygous “wild-type” IDH1 or IDH2 sequence (see, e.g., NCBI gene 3417 and RefSeq NM_005896 for IDH1, and NCBI gene 3418 and RefSeq NM_002168 for IDH2). A “reference” also includes value, amount, sequence, or other characteristic obtained from one or more control tissues, for example, a wild-type IDH1/2 homozygous tissue (cancerous or non-cancerous) from one or more controls, for example, one or more control subjects (e.g., a population of control subjects). As with the cancer tissue, the IDH1/2 mutation status from a control can be determined by any variety of methods, including, for example, ISH, FISH, WES, SNP array, NGS, or CGH on a human IDH1/2 protein or gene. Also as above, the D-2HG levels from a control can be determined by any variety of methods, including, for example, by biochemical detection (e.g., colorimetric assays), biosensors, GC- or LC-MS, MALDI-TOF, and others (supra).

In some embodiments, the tissue sample is a liquid biopsy (for example, a blood sample), a surgical sample, or other biopsy sample obtained from the subject. In specific embodiments, the tissue sample is a cancer tissue sample. Certain embodiments include the step of obtaining the tissue sample from the subject, for example, prior to determining IDH1 or IDH2 mutation status and/or D-2HG levels. In some embodiments, the subject is a human subject.

The methods provided herein can be performed on a variety of cancer types. In certain embodiments, the cancer is selected from glioma (including low-grade glioma, and high-grade glioma), medulloblastoma, chondrosarcoma, cholangiocarcinoma, acute myeloid leukemia (AML), astrocytoma, sinonasal undifferentiated carcinoma (SNUC), angioimmunoblastic T cell lymphoma (AITL), and glioblastoma (optionally secondary glioblastoma).

Certain embodiments include combination therapies for treating cancers, including methods of treating ameliorating the symptoms of, or inhibiting the progression of, a cancer in a subject in need thereof, comprising administering to the subject zotiraciclib in combination with at least one additional agent, for example, an immunotherapy agent (e.g., checkpoint inhibitor), a chemotherapeutic agent, a hormonal therapeutic agent, and/or a kinase inhibitor. In some embodiments, administering the zotiraciclib enhances the susceptibility of the cancer to the additional agent (for example, immunotherapy agent, chemotherapeutic agent, hormonal therapeutic agent, and or kinase inhibitor) by about or at least about 5, 10, 15, 20, 25, 30, 35, 40, 45, 50, 60, 70, 80, 90, 100, 200, 300, 400, 500, 600, 700, 800, 900, 1000, 2000% or more relative to the additional agent alone.

Certain combination therapies employ one or more chemotherapeutic agents, for example, small molecule chemotherapeutic agents. Non-limiting examples of chemotherapeutic agents include IDH1 and IDH2 inhibitors, alkylating agents, anti-metabolites, cytotoxic antibiotics, topoisomerase inhibitors (type 1 or type II), an anti-microtubule agents, among others.

Examples of IDH1 or IDH2 inhibitors include ivosidenib (AG-120), enasidenib, and AG-221 (see, for example, Zarei et al., Cancer Treat Rev. 103:102334, 2022. doi: 10.1016/j.ctrv.2021.102334; Hansen et al., Blood. 124 (21): 3734, 2014; Quivoron et al., Blood. 124 (21): 3735, 2014)

Examples of alkylating agents include nitrogen mustards (e.g., mechlorethamine, cyclophosphamide, mustine, melphalan, chlorambucil, ifosfamide, and busulfan), nitrosoureas (e.g., N-Nitroso-N-methylurea (MNU), carmustine (BCNU), lomustine (CCNU), semustine (MeCCNU), fotemustine, and streptozotocin), tetrazines (e.g., dacarbazine, mitozolomide, and temozolomide), aziridines (e.g., thiotepa, mytomycin, and diaziquone (AZQ)), cisplatins and derivatives thereof (e.g., carboplatin and oxaliplatin), and non-classical alkylating agents (optionally procarbazine and hexamethylmelamine).

Examples of anti-metabolites include anti-folates (e.g., methotrexate and pemetrexed), fluoropyrimidines (e.g., 5-fluorouracil and capecitabine), deoxynucleoside analogues (e.g., ancitabine, enocitabine, cytarabine, gemcitabine, decitabine, azacitidine, fludarabine, nelarabine, cladribine, clofarabine, fludarabine, and pentostatin), and thiopurines (e.g., thioguanine and mercaptopurine);

Examples of cytotoxic antibiotics include anthracyclines (e.g., doxorubicin, daunorubicin, epirubicin, idarubicin, pirarubicin, aclarubicin, and mitoxantrone), bleomycins, mitomycin C, mitoxantrone, and actinomycin. Examples of topoisomerase inhibitors include camptothecin, irinotecan, topotecan, etoposide, doxorubicin, mitoxantrone, teniposide, novobiocin, merbarone, and aclarubicin.

Examples of anti-microtubule agents include taxanes (e.g., paclitaxel and docetaxel) and vinca alkaloids (e.g., vinblastine, vincristine, vindesine, vinorelbine).

Certain combination therapies employ at least one hormonal therapeutic agent. General examples of hormonal therapeutic agents include hormonal agonists and hormonal antagonists. Particular examples of hormonal agonists include progestogen (progestin), corticosteroids (e.g., prednisolone, methylprednisolone, dexamethasone), insulin like growth factors, VEGF derived angiogenic and lymphangiogenic factors (e.g., VEGF-A, VEGF-A145, VEGF-A165, VEGF-C, VEGF-D, PIGF-2), fibroblast growth factor (FGF), galectin, hepatocyte growth factor (HGF), platelet derived growth factor (PDGF), transforming growth factor (TGF)-beta, androgens, estrogens, and somatostatin analogs. Examples of hormonal antagonists include hormone synthesis inhibitors such as aromatase inhibitors and gonadotropin-releasing hormone (GnRH) s agonists (e.g., leuprolide, goserelin, triptorelin, histrelin) including analogs thereof. Also included are hormone receptor antagonist such as selective estrogen receptor modulators (SERMs; e.g., tamoxifen, raloxifene, toremifene) and anti-androgens (e.g., flutamide, bicalutamide, nilutamide).

Also included are hormonal pathway inhibitors such as antibodies directed against hormonal receptors. Examples include inhibitors of the IGF receptor (e.g., IGF-IR1) such as cixutumumab, dalotuzumab, figitumumab, ganitumab, istiratumab, and robatumumab; inhibitors of the vascular endothelial growth factor receptors 1, 2 or 3 (VEGFR1, VEGFR2 or VEGFR3) such as alacizumab pegol, bevacizumab, icrucumab, ramucirumab; inhibitors of the TGF-beta receptors R1, R2, and R3 such as fresolimumab and metelimumab; inhibitors of c-Met such as naxitamab; inhibitors of the EGF receptor such as cetuximab, depatuxizumab mafodotin, futuximab, imgatuzumab, laprituximab emtansine, matuzumab, modotuximab, necitumumab, nimotuzumab, panitumumab, tomuzotuximab, and zalutumumab; inhibitors of the FGF receptor such as aprutumab ixadotin and bemarituzumab; and inhibitors of the PDGF receptor such as olaratumab and tovetumab.

Certain combination therapies employ at least one kinase inhibitor, including tyrosine kinase inhibitors. Examples of kinase inhibitors include, without limitation, adavosertib, afatinib, aflibercept, axitinib, bevacizumab, bosutinib, cabozantinib, cetuximab, cobimetinib, crizotinib, dasatinib, entrectinib, erdafitinib, erlotinib, fostamatinib, gefitinib, ibrutinib, imatinib, lapatinib, lenvatinib, mubritinib, nilotinib, panitumumab, pazopanib, pegaptanib, ponatinib, ranibizumab, regorafenib, ruxolitinib, sorafenib, sunitinib, SU6656, tofacitinib, trastuzumab, vandetanib, and vemurafenib.

In some embodiments, the methods and pharmaceutical compositions described herein increase median survival time of a subject by 4 weeks, 5 weeks, 6 weeks, 7 weeks, 8 weeks, 9 weeks, 10 weeks, 15 weeks, 20 weeks, 25 weeks, 30 weeks, 40 weeks, or longer. In some embodiments, the methods and compositions described herein increase progression-free survival, overall survival, and/or survival post-progression in the subject in need thereof, for example, by about or at least about 2, 3, 4, 5, 6, 7, 8, 9, 10, 11, 12, 13, 14, 15, 16, 17, 18, 19, 20, 21, 22, 23, or 24 months or more, or by about or at least about 1, 2, 3, 4, 5, 6, 7, 8, 9, or 10 years or more. In certain embodiments, the methods and compositions described are sufficient to result in stable disease.

In certain embodiments, the methods and therapeutic compositions described herein are sufficient to result in tumor regression, as indicated by a statistically significant decrease in the amount of viable tumor, for example, at least a 10%, 20%, 30%, 40%, 50% or greater decrease in tumor mass, or by altered (e.g., decreased with statistical significance) scan dimensions. In certain embodiments, the methods and therapeutic compositions described herein are sufficient to result in stable disease.

In certain embodiments, the methods and therapeutic compositions described herein are sufficient to result in clinically relevant reduction in symptoms of a particular disease indication known to the skilled clinician.

The methods for treating cancers can be combined with other therapeutic modalities. For example, a cancer therapy described herein can be administered to a subject before, during, or after other therapeutic interventions, including symptomatic care, radiation therapy (radiotherapy), surgery, transplantation, hormone therapy, photodynamic therapy, antibiotic therapy, or any combination thereof. Symptomatic care includes administration of corticosteroids, to reduce cerebral edema, headaches, cognitive dysfunction, and emesis, and administration of anti-convulsants, to reduce seizures. Radiotherapy includes whole-brain irradiation, fractionated radiotherapy, and radiosurgery, such as stereotactic radiosurgery, which can be further combined with traditional surgery. In some embodiments, the radiotherapy includes administering a total radiation dose of about 1 Gray (Gy) to about 70 Gy, or about 1, 2, 3, 4, 5, 6, 7, 8, 9, 10, 15, 20, 25, 30, 35, 40, 45, 50, 55, 60, 65, or 70 Gy.

Methods for identifying subjects with one or more of the diseases or conditions described herein are known in the art.

For in vivo use, for instance, for the treatment of human disease or testing, the agents described herein are generally incorporated into one or more therapeutic or pharmaceutical compositions prior to administration.

To prepare a therapeutic or pharmaceutical composition, an effective or desired amount of one or more agents is typically mixed with any pharmaceutical carrier(s) or excipient known to those skilled in the art to be suitable for the particular agent and/or mode of administration. A pharmaceutical carrier may be liquid, semi-liquid or solid. Solutions or suspensions used for parenteral, intradermal, subcutaneous or topical application may include, for example, a sterile diluent (such as water), saline solution (e.g., phosphate buffered saline; PBS), fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously (e.g., by IV infusion), suitable carriers include physiological saline or phosphate buffered saline (PBS), and solutions containing thickening and solubilizing agents, such as glucose, polyethylene glycol, polypropylene glycol and mixtures thereof.

Administration of agents described herein, in pure form or in an appropriate therapeutic or pharmaceutical composition, can be carried out via any of the accepted modes of administration of agents for serving similar utilities. The therapeutic or pharmaceutical compositions can be prepared by combining an agent-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other small molecules as described elsewhere herein) and/or suitable excipients such as salts, buffers and stabilizers may, but need not, be present within the composition.

Administration may be achieved by a variety of different routes, including oral, parenteral, nasal, intravenous, intradermal, intramuscular, subcutaneous or topical. Preferred modes of administration depend upon the nature of the condition to be treated or prevented. Particular embodiments include administration by IV infusion.

Carriers can include, for example, pharmaceutically- or physiologically-acceptable carriers, excipients, or stabilizers that are non-toxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

In some embodiments, one or more agents can be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980). The particle(s) or liposomes may further comprise other therapeutic or diagnostic agents.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

Typical routes of administering these and related therapeutic or pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Therapeutic or pharmaceutical compositions according to certain embodiments of the present disclosure are formulated so as to allow the active ingredients contained therein to be bioavailable upon administration of the composition to a subject or patient. Compositions that will be administered to a subject or patient may take the form of one or more dosage units, where for example, a tablet may be a single dosage unit, and a container of a herein described agent in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will typically contain a therapeutically effective amount of an agent described herein, for treatment of a disease or condition of interest.

A therapeutic or pharmaceutical composition may be in the form of a solid or liquid. In one embodiment, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid. Certain embodiments include sterile, injectable solutions.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The therapeutic or pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid therapeutic or pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid therapeutic or pharmaceutical composition intended for either parenteral or oral administration should contain an amount of an agent such that a suitable dosage will be obtained. Typically, this amount is at least 0.01% of the agent of interest in the composition. When intended for oral administration, this amount may be varied to be between 0.1 and about 70% of the weight of the composition. Certain oral therapeutic or pharmaceutical compositions contain between about 4% and about 75% of the agent of interest. In certain embodiments, therapeutic or pharmaceutical compositions and preparations according to the present invention are prepared so that a parenteral dosage unit contains between 0.01 to 10% by weight of the agent of interest prior to dilution.

The therapeutic or pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The therapeutic or pharmaceutical compositions in solid or liquid form may include a component that binds to agent and thereby assists in the delivery of the compound. Suitable components that may act in this capacity include monoclonal or polyclonal antibodies, one or more proteins or a liposome.

The compositions described herein may be prepared with carriers that protect the agents against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

The therapeutic or pharmaceutical compositions may be prepared by methodology well known in the pharmaceutical art. For example, a therapeutic or pharmaceutical composition intended to be administered by injection may comprise one or more of salts, buffers and/or stabilizers, with sterile, distilled water so as to form a solution. A surfactant may be added to facilitate the formation of a homogeneous solution or suspension. Surfactants are compounds that non-covalently interact with the agent so as to facilitate dissolution or homogeneous suspension of the agent in the aqueous delivery system.

Certain embodiments include the use of a diagnostic kit for treating a cancer in a human subject in need thereof with zotiraciclib (TG02), or an analog, derivative, or pharmaceutically acceptable salt thereof, comprising means for determining isocitrate dehydrogenase (IDH1 or IDH2) mutation status in a tissue sample from the subject. Also included are patient care kits, comprising: (a) means for determining isocitrate dehydrogenase (IDH1 or IDH2) mutation status in a tissue sample from a human subject with cancer; and (b) a composition comprising zotiraciclib (TG02), or an analog, derivative, or pharmaceutically acceptable salt thereof.

In some embodiments, the means for (directly) determining IDH1 or IDH2 mutation status in a tissue sample comprise reagents for performing a diagnostic assay selected from one or more of DNA or RNA sequencing, ISH, FISH, WES, SNP array, NGS, or CGH on a human IDH1 or IDH2 protein or gene. In certain embodiments, the means for (indirectly) determining IDH1 or IDH2 mutation status in a tissue sample comprise reagents for performing a diagnostic assay to determine D-2HG levels in a tissue sample, for example, a diagnostic assay selected from biochemical detection (e.g., colorimetric assays), biosensors, GC- or LC-MS, and MALDI-TOF.

Some diagnostic or patient care kits include an IDH1/2 gene reference obtained from a database, or determined from a control or reference, for example, a homozygous wild-type IDH1/2 control. The kits can also include written instructions, for example, on how to determine or measure IDH1/2 mutation status, and/or the levels, presence, absence of D-2HG in a tissue sample from a subject and/or from a control.

Certain diagnostic or patient care kits comprise one or more additional agents, for example, immunotherapy agents, chemotherapeutic agents, hormonal therapeutic agents, and/or kinase inhibitor, as described herein.

In some embodiments, a diagnostic or patient care kit contains separate containers, dividers, or compartments for the composition(s) and informational material(s). For example, the composition(s) or reagents can be contained in a bottle, vial, or syringe, and the informational material(s) can be contained in association with the container. In some embodiments, the separate elements of the kit are contained within a single, undivided container. For example, the composition(s) or reagents are contained in a bottle, vial or syringe that has attached thereto the informational material in the form of a label. In some embodiments, the kit includes a plurality (e.g., a pack) of individual containers, each containing one or more compositions, reagents, and/or unit dosage forms of zotiraciclib. For example, the kit includes a plurality of syringes, ampules, foil packets, or blister packs, each containing a reagent or a single unit dose of zotiraciclib. The containers of the kits can be air tight, waterproof (e.g., impermeable to changes in moisture or evaporation), and/or light-tight.

The patient care kit optionally includes a device suitable for administration of the agent(s), e.g., a syringe, inhalant, dropper (e.g., eye dropper), swab (e.g., a cotton swab or wooden swab), or any such delivery device. In some embodiments, the device is an implantable device that dispenses metered doses of the agent(s). Also included are methods of providing a kit, e.g., by combining the components described herein.

In certain aspects, the diagnostic or therapeutic response tests or methods described herein are performed at a diagnostic laboratory, and the results are then provided to the subject, or to a physician or other healthcare provider that plays a role in the subject's healthcare and cancer treatment. Particular embodiments thus include methods for providing the results of the responsiveness test to the subject in need thereof, or to the physician or other healthcare provider. These results or data can be in the form of a hard-copy or paper-copy, or an electronic form, such as a computer-readable medium.

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill certain changes and modifications may be made thereto without departing from the spirit or scope of the description or appended claims. The following examples are provided by way of illustration only and not by way of limitation. Those of skill in the art will readily recognize a variety of noncritical parameters that could be changed or modified to yield essentially similar results.

### EXAMPLES

### Example 1

Experiments were performed to test the activity of zotiraciclib (TG02) against IDH1 mutant cancer cells, and to evaluate the mechanism of such activity.

### Materials & Methods

Cell Culture. Human Chondrosarcoma cell line HT-1080 was cultured in MEM (Gibco, USA) supplemented with 10% fetal bovine serum (FBS, Gemini, USA). Human cholangiocarcinoma cell lines RBE, Hccc-9810, and Hucct-1 were cultured in RPMI1640 (Hyclone, USA) supplemented with 10% FBS. Cells were incubated at 37 °C in 5% CO₂.

Apoptosis Assay. HT-1080, RBE, Hccc-9810, and Hucct-1 cells were treated with the indicated drugs. Apoptosis was detected by Annexin V/PI staining kit (Thermo Fisher) according to the manufacturer's instructions. Briefly, cells were plated at a density of 1× 10⁵ cells/well in media with 10% FBS with desired concentrations of TG02. At 48/72-hour post-treatment the cells were harvested and tested for apoptosis by Annexin V and PI staining. Cell analysis was performed using a FACSCelesta (BD).

2Hydroxyglutarate (D-2HG) assay. Cells (~1×10⁷) were rapidly homogenized with 100 μl ice cold D-2HG Assay buffer in D2Hydroxyglutarate Assay Kit (Abcam, ab211070) for 10 min on ice. Cells were then centrifuged at 10000×g, 4 °C for 5 min, the supernatant was collected, and the same volume of each sample was added into three wells of a 96 well clear plate.

The 100 Mm D-2HG standard was diluted to 1 mM (1 nmol/μl) by adding 10 μl of 100 mM D-2HG standard solution to 990 μl D-2HG Assay Buffer and mixed well.

Five μl of 1 mM D-2HG Standard was added to one of three Samples defined as: Spiked Sample (5 nmol D2Hydroxyglutarate+Sample); Sample; and Sample Background. The Spiked Sample was used as an Internal Standard to correct for any Sample interference. The final volume of all wells was adjusted to 50 μl with D-2HG Assay Buffer.

For each well, 50 μl Reaction Mix was prepared (see Table E1):

TABLE E1

| Item | Reaction Mix | Background Control Mix |
|---|---|---|
| D-2HG Assay Buffer | 46 μl | 48 μl |
| D-2HG Enzyme | 2 μl | 0 μl |
| D-2HG Substrate Mix | 2 μl | 2 μl |

50 μl of the Reaction Mix was added to each well containing the Standards and Samples, and mixed well. The plate was incubated for 60 min at 37 °C and the OD450 nm was measured.

The Sample Background reading was subtracted from its paired Sample reading to get the Sample Corrected reading. The D-2HG amount in the Sample wells (X) was determined based on the following equation:

D-2HG amount(nmol)=(OD sample(corrected)/[(OD(spiked sample))−(OD sample)]×5

Comet assay. The comet assay is common technique for measurement of DNA damage in individual cells. Under an electrophoretic field, damaged cellular DNA (containing fragments and strand breaks) is separated from intact DNA, yielding a classic “comet tail” shape under the microscope. DNA damage was assayed by OxiSelect™ Comet Assay Kit (CELL BIOLABS, INC.) according to the manufacturer's instructions. Briefly, comet agarose was pipetted onto the OxiSelect™ Comet Slide to form a base layer. Cells were combined with OxiSelect™ Comet agarose at 37 °C, then the agarose/cell mixture was pipetted onto the top of the base layer. Cells were treated with lysis buffer and alkaline solution. Electrophoresis was performed under neutral conditions. The slides were viewed by epifluorescence microscopy using a FITC filter.

Tail Moment=(100×Tail DNA Intensity/Cell DNA Intensity)×Tail Moment Length

Western blot. Hccc-9810 and Hucct-1 cells treated with TG02 or AG120 as indicated for 48 h (0.1% DMSO was added as control) were harvested and centrifuged at 500 g for 5 minutes to obtain cell pellets. Then the pellets were lysed in lysis buffer (Beyotime, China) which additionally included a protease inhibitor cocktail (Beyotime, China) and phosphatase inhibitor cocktail (Beyotime, China). Cells were incubated on ice for 30 minutes and then centrifuged at 4 °C, 12000 rpm for 10 minutes to obtain the supernatant as cell lysate. The concentrations of protein in cell lysate were determined by Micro BCA™ Protein Assay Kit (ThermoFisher, USA). 4×SDS-PAGE Sample Loading Buffer (SolarBio, China) was added into cell lysate contained 25 μg of total protein and, after boiling, the mixture was electrophoresed in polyacrylamide gel. After electrophoresis, proteins on the gel were transferred to PVDF membrane, and the membrane was cut at position close to the molecular weights of proteins whose expression was examined (pH2AX (CST, USA), GAPDH (ZSGB-BIO, China)).

Transfection. The day before transfection, cells (~2×10⁵/well) were seeded in a 6-well plate. The cells were cultured in a 37 °C in 5% CO₂ incubator overnight. The old medium was removed and 2 ml of transfection medium was added. 50 μl opti-MEM was added to a tube, and then 2 μg plasmid DNA Mix solution was mixed by vortexing. 4 μl PEI solution was added to this tube, and the solution was mixed by gently pipetting up and down. The samples were incubated at room temperature for 15 min to allow the PEI/DNA complexes to form. The PEI/DNA complexes were gently added dropwise to well containing the cells. The cells were mixed gently by swirling, and incubated for 48 h in a 37 °C, 5% CO2. The cells were then used to detect the drug efficacy or gene expression.

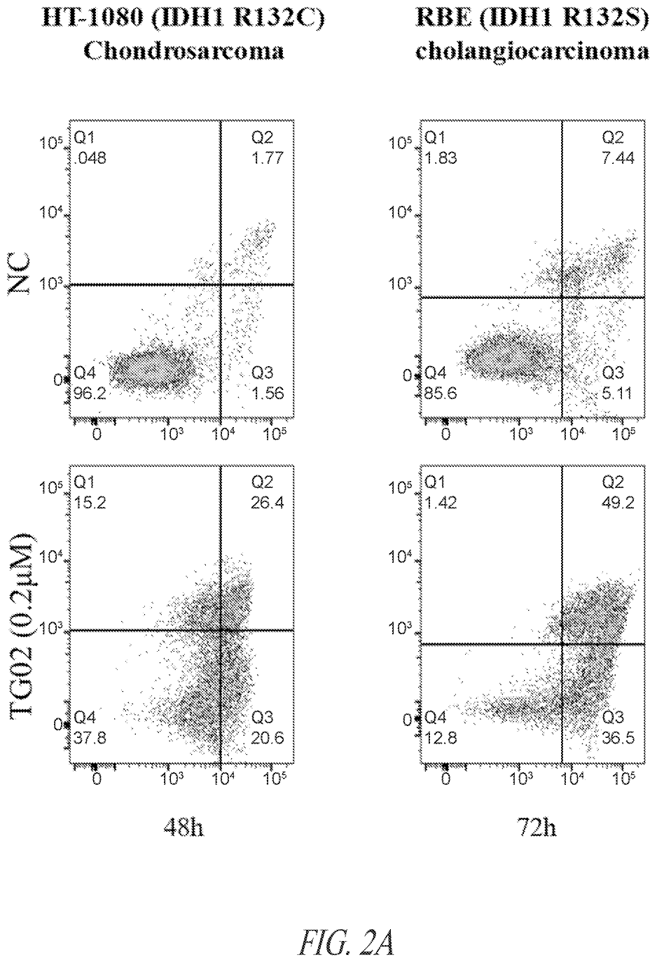

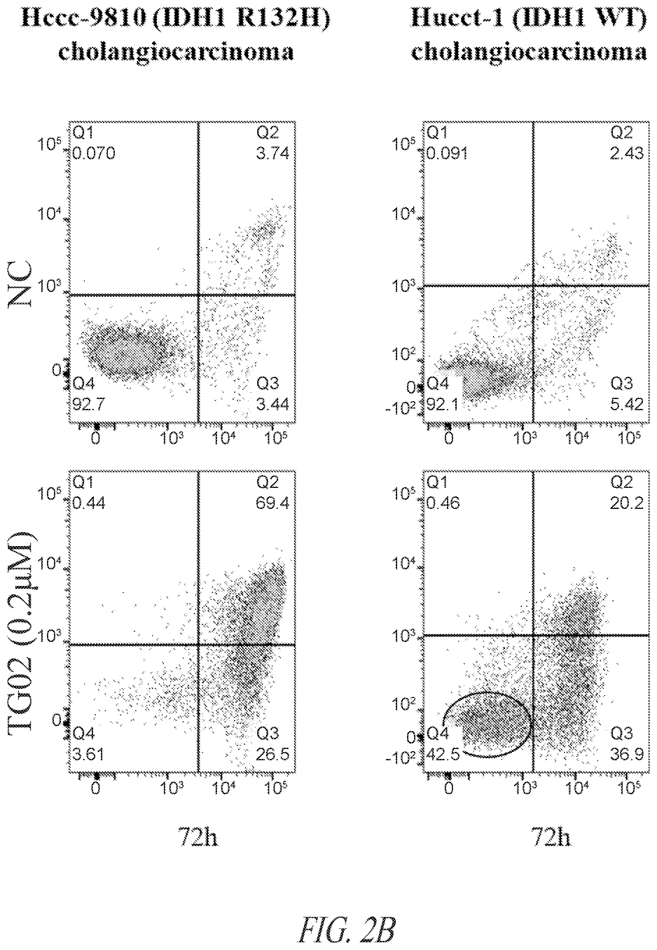

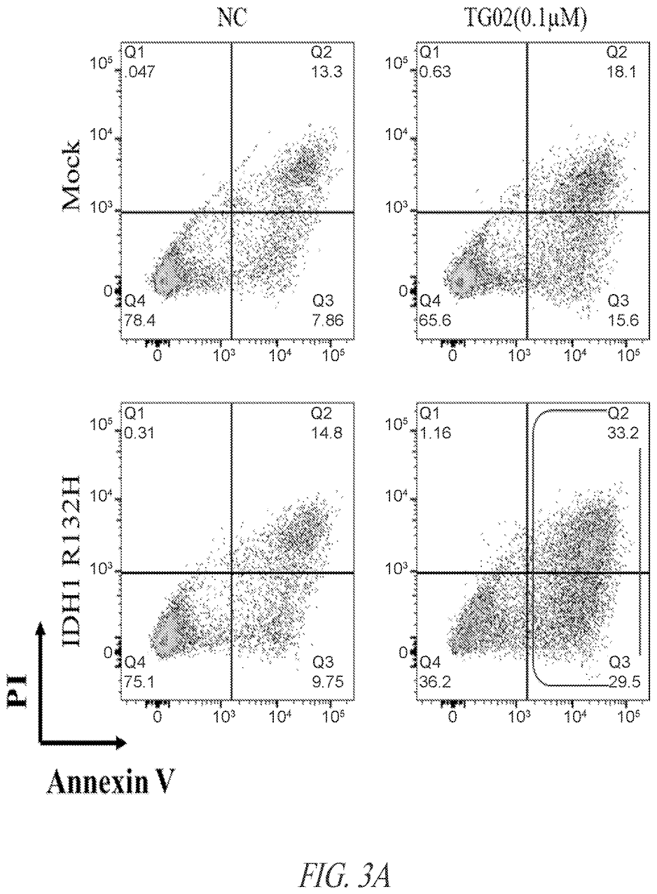

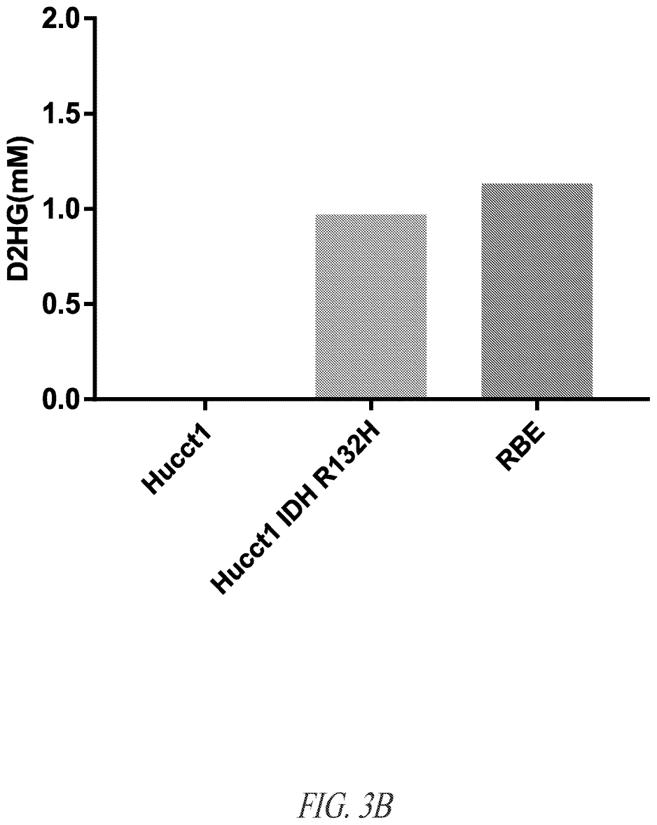

Results. As shown in FIGS. 2A-2B, the IDH1-mutant chondrosarcoma (HT-1080) and cholangiocarcinoma (RBE, Hcc-9810) cells were more sensitive to TG02 than IDH1-WT (Hucct-1) cells. FIGS. 3A-3B shows that increased TG02 sensitivity can be conferred onto IDH1-WT (Hucct-1) cells by transfection of mutated IDH1. In FIG. 3A, Hucct-1 cells were transfected with IDH1 R132H plasmid and treated with 0.1 μM TG02 for 72 h. Apoptosis was determined by FACS assay. In FIG. 3B, Hucct-1 cells were transfected with IDH1 R132H plasmid, and the levels of D-2-hydroxyglutarate (D-2HG) were measured in Hucct-1 WT, Hucct-1 transfected with IDH1 R132H plasmid, and RBE (cholangiocarcinoma cell line with IDH1 R132S mutation) cells. D-2HG is a metabolic biomarker of gain-of-function mutation(s) in the IDH1 and/or IDH2 genes.

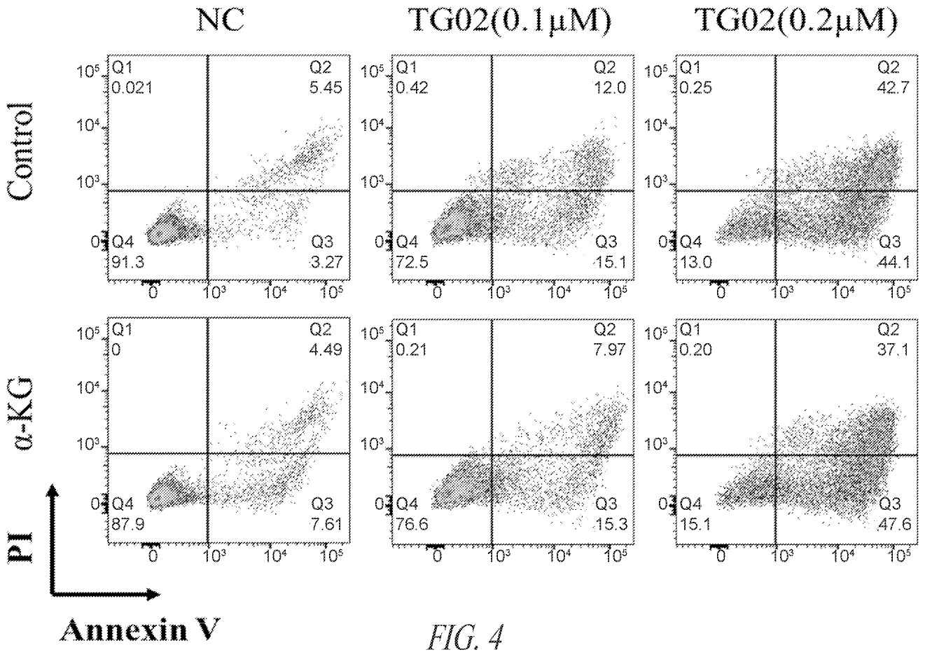

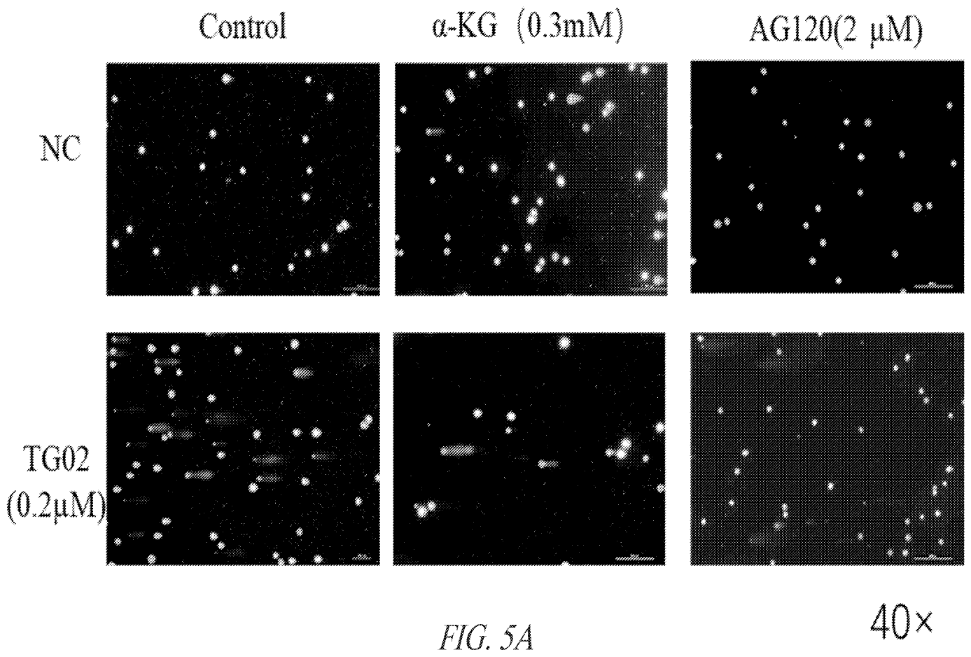

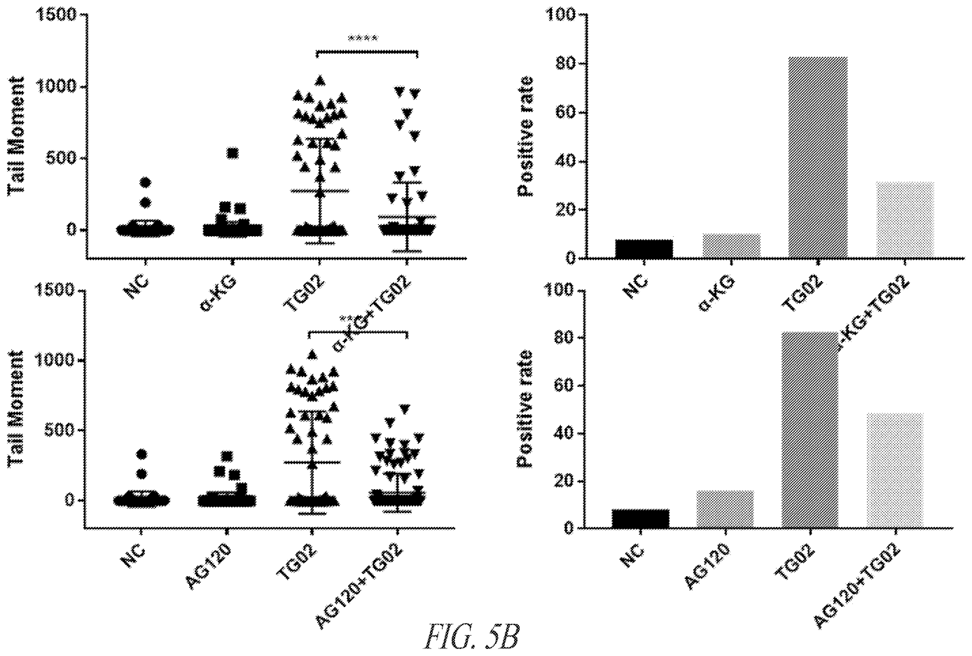

FIG. 4 shows that TG02-induced apoptosis of mutant IDH1 (Hccc-9810) cells can be rescued by addition of exogenous substrate α-ketoglutarate (α-KG). Mutant IDH1/2 converts α-KG into the oncometabolite (D-2HG), which then inhibits a class of α-KG-dependent enzymes involved in epigenetic regulation. Cells were pretreated with 24 h with α-KG for 24 h, then treated with TG02 for 72 h. Apoptosis was determined by FACS assay. Likewise, FIGS. 5A-5B show that TG02-induced DNA damage in mutant IDH1 (HT-1080) cells can be rescued by addition of exogenous α-KG or AG-120. AG-120 is an inhibitor of mutant IDH1. In FIG. 5A, cells were treated with indicated drugs for 48 h, and DNA damage was determined by the neutral comet assay. FIG. 5B shows statistical analyses of the data in 5A (Left panels: Tail moment. Right panels: positive percent of cells with DNA damage).

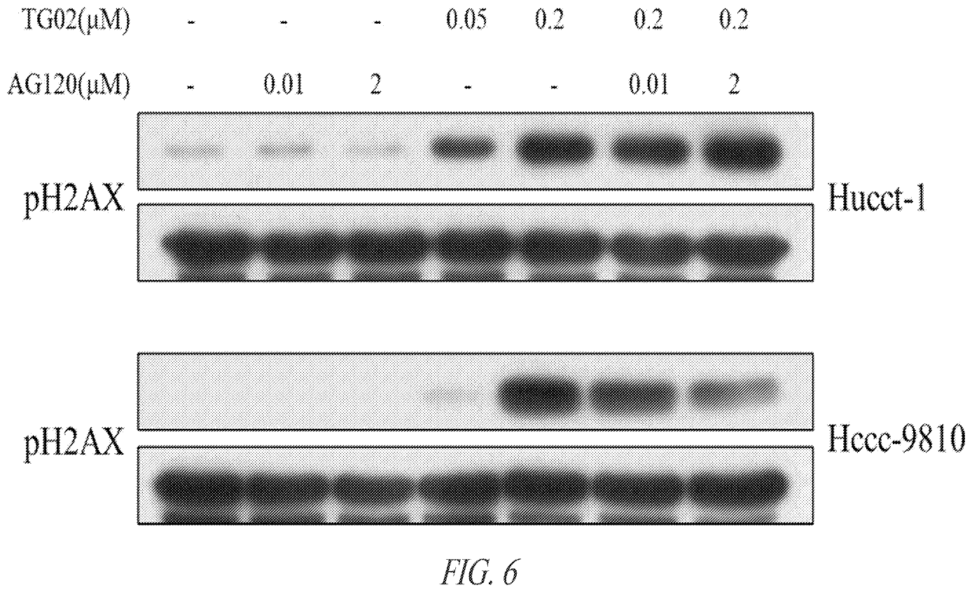

FIG. 6 shows that TG02-induced DNA damage was rescued only in mutant IDH1 cells (Hccc-9810) by addition of exogenous AG-120. Cells were treated with indicated drugs for 48 h. pH2AX, the marker of DNA damage, was assayed by WB

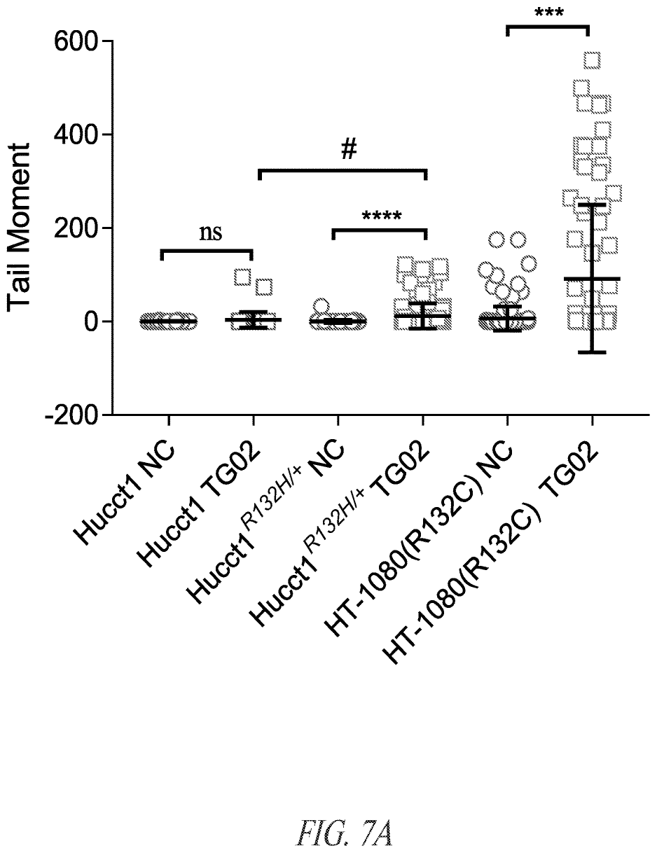

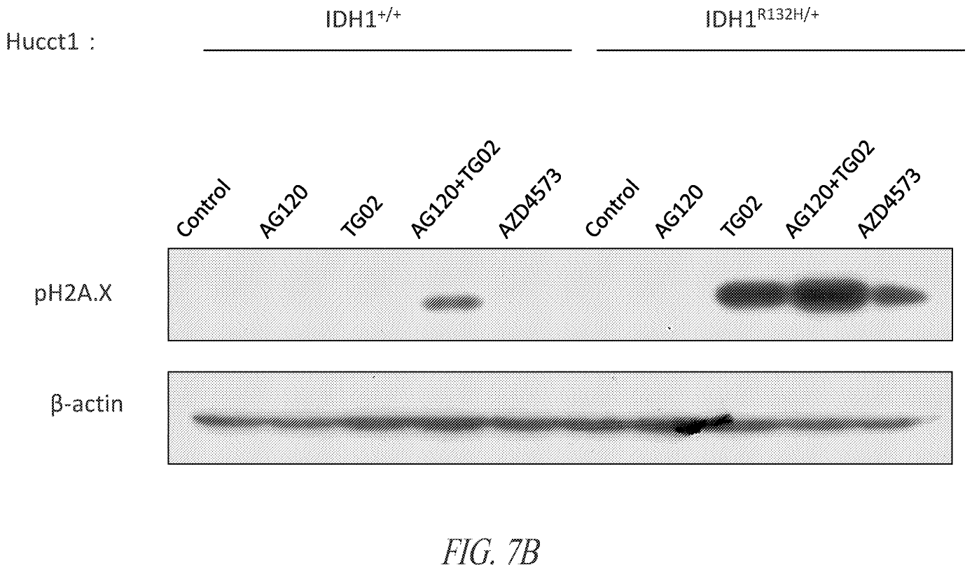

FIGS. 7A-7B show that cells with IDH1 mutation are more sensitive to DNA damage induced by TG02. In FIG. 7A, IDH1-mutant Hucct1 cells (Hucct1^{R132H/+}) were generated by lentivirus expressing IDH1 R132H. Hucct1, Hucct1^{R132H/+} and HT-1080 cells were treated with 0.2 μM TG02 or 0.1% DMSO for 48 h. Cells were used to comet assays. Quantification of Tail moment in neutral comet assay is presented. ns: no significant differences; ***: P<0.001, ****: P<0.0001, compared with NC; #: P<0.05, compared with Hucct1 cells treated with TG02. In FIG. 7B, Hucct1 and Hucct1R132H/+ cells were treated with 2 μM AG120, 0.2 μM TG02 or 0.01 μM AZD4573 (CDK9 inhibitor) as indicated. Shown is Western blot analysis of phosphorylated γH2AX (a marker of DSBs) after being treated for 16 h. β-actin was used as a loading control.

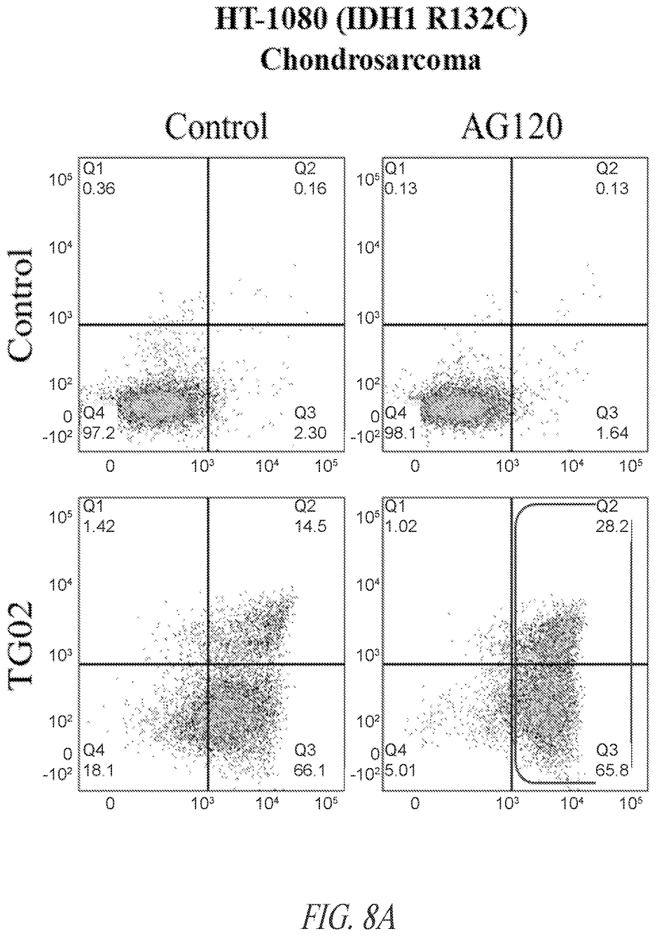

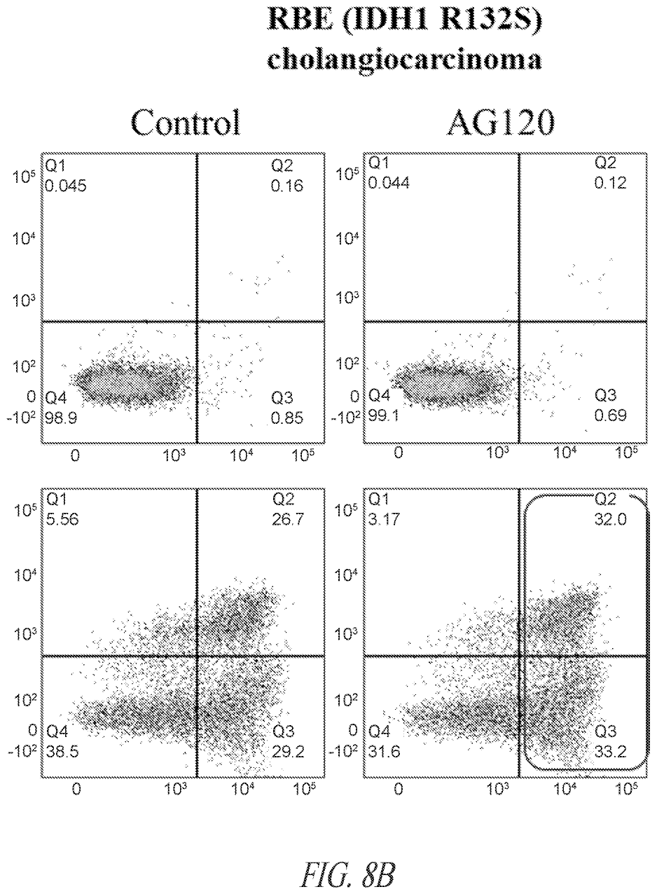

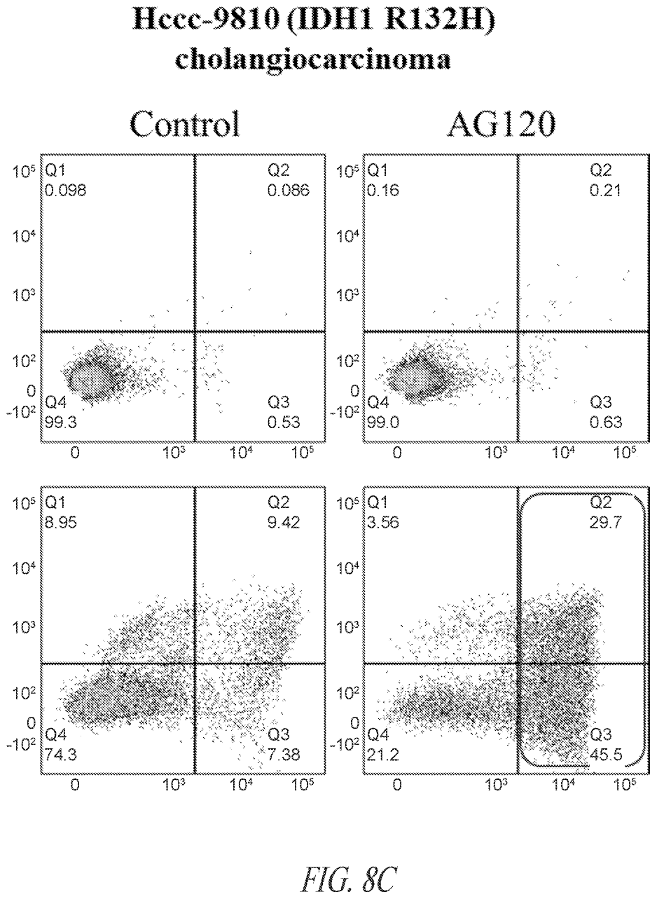

FIGS. 8A-8C show that TG02 combined with AG120 induced cell apoptosis in cells with mutant IDH1. Mutant IDH1 cells HT-1080 (8A), RBE (8B), and Hccc-9810 (8C) were treated with TG02 (0.1 μM) and AG120 (2 μM) as indicated for 48 h, and apoptosis was measured by Annexin V/PI staining and analysis by flow cytometry.

The evidence thus shows that TG02 selectively induces DNA damage and cell-killing in mutant IDH1 cancer cells relative to homozygous wild-type IDH1 cancer cells. The evidence also shows that such TG02-sensitivity is related to the ‘gain-of-function’, cancer-promoting activity of mutant IDH1 (shared by mutant IDH2), which converts α-KG into the oncometabolite D-2HG.

1. A method for treating a cancer in a human subject in need thereof, wherein the cancer comprises an isocitrate dehydrogenase (IDH1 or IDH2) mutation, comprising
administering to the subject a composition comprising zotiraciclib (TG02), or a pharmaceutically acceptable salt thereof,
thereby treating the cancer comprising the IDH1 or IDH2 mutation.

2. The method of claim 1, wherein the cancer is selected from glioma (optionally low-grade or high-grade glioma), medulloblastoma, chondrosarcoma, cholangiocarcinoma, acute myeloid leukemia (AML), astrocytoma, sinonasal undifferentiated carcinoma (SNUC), angioimmunoblastic T cell lymphoma (AITL), and glioblastoma (optionally secondary glioblastoma).

3. The method of claim 1 or 2, comprising:
(a) determining IDH1 or IDH2 mutation status in a tissue sample from the subject; and
(b) administering to the subject a composition comprising zotiraciclib (TG02), or the pharmaceutically acceptable salt thereof, if the tissue sample comprises the IDH1 or IDH2 mutation.

4. The method of any one of claims 1-3, wherein the IDH1 or IDH2 mutation is a gain-of-function mutation characterized by increased conversion of α-ketoglutarate (α-KG) into an oncometabolite D-2-hydroxyglutarate (D-2HG) relative to homozygous wild-type IDH1 or IDH2.

5. The method of any one of claims 1-4, wherein the IHD1 mutation is R132X, wherein X is selected from any amino acid other than R, optionally wherein the IHD1 mutation is R132C, R132G, R132H, R132L, or R132S.

6. The method of any one of claims 1-4, wherein the IHD2 mutation is R172X or R140X, wherein X is selected from any amino acid other R, optionally wherein the IHD2 mutation is R172G, R172K, R172M, R172S, R172T, or R140Q.

7. The method of any one of claims 3-6, wherein (a) comprises determining IDH1 or IDH2 mutation status in the tissue sample by DNA or RNA sequencing, in situ hybridization (ISH), fluorescence in situ hybridization (FISH), whole exome sequencing (WES), single nucleotide polymorphism (SNP) array, next generation sequencing (NGS), or comparative genome hybridization (CGH) on a human IDH1 or IDH2 protein or gene.

8. The method of any one of claims 3-7, comprising obtaining the tissue sample from the subject.

9. The method of any one of claims 3-8, wherein the tissue sample is a liquid biopsy optionally a blood sample, a surgical sample, or other biopsy sample obtained from the subject, optionally wherein the tissue sample is a cancer tissue sample.

10. The method of any one of claims 1-9, comprising administering to the subject an oral composition of zotiraciclib, or a pharmaceutically acceptable salt thereof.

11. The method of any one of claims 1-10, comprising administering the composition comprising zotiraciclib in combination with radiotherapy and/or one or more additional agents, optionally selected from chemotherapeutic agents, hormonal therapeutic agents, and/or kinase inhibitors.

12. The method of claim 11, wherein the chemotherapeutic agent comprises an IDH1 or IDH2 inhibitor, optionally ivosidenib (AG-120), enasidenib, or AG-221.

13. Use of a diagnostic kit for treating a cancer in a human subject in need thereof with zotiraciclib (TG02), or a pharmaceutically acceptable salt thereof, wherein the cancer comprises an isocitrate dehydrogenase (IDH1 or IDH2) mutation, comprising means for determining isocitrate dehydrogenase (IDH1 or IDH2) mutation status in a tissue sample from the subject.

14. The use of claim 13, wherein the IDH1 or IDH2 mutation is a gain-of-function mutation characterized by increased conversion of α-ketoglutarate (α-KG) into an oncometabolite D-2-hydroxyglutarate (D-2HG) relative to homozygous wild-type IDH1 or IDH2.

15. The use of claim 13 or 14, wherein the IHD1 mutation is R132X, wherein X is selected from any amino acid other than R, optionally wherein the IHD1 mutation is R132C, R132G, R132H, R132L, or R132S.

16. The use of any one of claims 13-15, wherein the IHD2 mutation is R172X or R140X, wherein X is selected from any amino acid other R, optionally wherein the IHD2 mutation is R172G, R172K, R172M, R172S, R172T, or R140Q.

17. The use of any one of claims 13-16, wherein the means for determining IDH1 or IDH2 mutation status in a tissue sample comprise reagents for performing a diagnostic assay selected from one or more of DNA or RNA sequencing, in situ hybridization (ISH), fluorescence in situ hybridization (FISH), whole exome sequencing (WES), single nucleotide polymorphism (SNP) array, next generation sequencing (NGS), or comparative genome hybridization (CGH) on a human IDH1 or IDH2 protein or gene.

18. The use of any one of claims 13-17, wherein the tissue sample is a liquid biopsy optionally a blood sample, a surgical sample, or other biopsy sample obtained from the subject, optionally a biopsy of prostate cancer tissue.

19. The use of any one of claims 13-18, wherein the cancer is selected from glioma (optionally low-grade or high-grade glioma), medulloblastoma, chondrosarcoma, cholangiocarcinoma, acute myeloid leukemia (AML), sinonasal undifferentiated carcinoma (SNUC), angioimmunoblastic T cell lymphoma (AITL), astrocytoma, and glioblastoma (optionally secondary glioblastoma).

20. The use of any one of claims 13-19, wherein the diagnostic kit comprises a composition comprising zotiraciclib, or a pharmaceutically acceptable salt thereof, optionally an oral composition of zotiraciclib.

21. The use of claim 20, wherein the diagnostic kit comprises one or more additional agents, optionally selected from chemotherapeutic agents, hormonal therapeutic agents, and/or kinase inhibitors.

22. The use of claim 21, wherein the chemotherapeutic agent comprises an IDH1 or IDH2 inhibitor, optionally ivosidenib (AG-120), enasidenib, or AG-221.

23. A patient care kit, comprising:
(a) means for determining isocitrate dehydrogenase (IDH1 or IDH2) mutation status in a tissue sample from a human subject with cancer; and
(b) a composition comprising zotiraciclib (TG02), or a pharmaceutically acceptable salt thereof.

24. The patient care kit of claim 23, wherein the IDH1 or IDH2 mutation is a gain-of-function mutation characterized by increased conversion of α-ketoglutarate (α-KG) into an oncometabolite D-2-hydroxyglutarate (D-2HG) relative to homozygous wild-type IDH1 or IDH2.

25. The patient care kit of claim 23 or 24, wherein the IHD1 mutation is R132X, wherein X is selected from any amino acid other than R, optionally wherein the IHD1 mutation is R132C, R132G, R132H, R132L, or R132S.

26. The patient care kit of any one of claims 23-25, wherein the IHD2 mutation is R172X or R140X, wherein X is selected from any amino acid other R, optionally wherein the IHD2 mutation is R172G, R172K, R172M, R172S, R172T, or R140Q.

27. The patient care kit of claims 13-16, wherein the means for determining IDH1 or IDH2 mutation status in a tissue sample comprise reagents for performing a diagnostic assay selected from one or more of DNA or RNA sequencing, in situ hybridization (ISH), fluorescence in situ hybridization (FISH), whole exome sequencing (WES), single nucleotide polymorphism (SNP) array, next generation sequencing (NGS), or comparative genome hybridization (CGH) on a human IDH1 or IDH2 protein or gene.

28. The patient care kit of any one of claims 23-27, wherein the tissue sample is a liquid biopsy optionally a blood sample, a surgical sample, or other biopsy sample obtained from the subject, optionally a biopsy of prostate cancer tissue.

29. The patient care kit of any one of claims 23-28, wherein the cancer is selected from glioma (optionally low-grade or high-grade glioma), medulloblastoma, chondrosarcoma, cholangiocarcinoma, acute myeloid leukemia (AML), astrocytoma, sinonasal undifferentiated carcinoma (SNUC), angioimmunoblastic T cell lymphoma (AITL), and glioblastoma (optionally secondary glioblastoma).

30. The patient care kit of any one of claims 23-29, wherein (b) comprises an oral composition of zotiraciclib, or a pharmaceutically acceptable salt thereof.

31. The patient care kit of any one of claims 23-30, wherein the patient care kit comprises one or more additional agents, optionally selected from chemotherapeutic agents, hormonal therapeutic agents, and/or kinase inhibitors.

32. The patient care kit of claim 11, wherein the chemotherapeutic agent comprises an IDH1 or IDH2 inhibitor, optionally ivosidenib (AG-120), enasidenib, or AG-221.

33. A pharmaceutical composition for use in a method of treating a cancer in a human subject in need thereof, wherein the cancer comprises an isocitrate dehydrogenase (IDH1 or IDH2) mutation, comprising zotiraciclib (TG02), or a pharmaceutically acceptable salt thereof.

34. The pharmaceutical composition for use according to claim 33, wherein the IDH1 or IDH2 mutation is a gain-of-function mutation characterized by increased conversion of α-ketoglutarate (α-KG) into an oncometabolite D-2-hydroxyglutarate (D-2HG) relative to homozygous wild-type IDH1 or IDH2.

35. The pharmaceutical composition for use according to claim 33 or 34, wherein the IHD1 mutation is R132X, wherein X is selected from any amino acid other than R, optionally wherein the IHD1 mutation is R132C, R132G, R132H, R132L, or R132S.

36. The pharmaceutical composition for use according to any one of claims 33-35, wherein the IHD2 mutation is R172X or R140X, wherein X is selected from any amino acid other R, optionally wherein the IHD2 mutation is R172G, R172K, R172M, R172S, R172T, or R140Q.

37. The pharmaceutical composition for use according to any one of claims 33-36, wherein the cancer is selected from glioma (optionally low-grade or high-grade glioma), medulloblastoma, chondrosarcoma, cholangiocarcinoma, acute myeloid leukemia (AML), astrocytoma, sinonasal undifferentiated carcinoma (SNUC), angioimmunoblastic T cell lymphoma (AITL), and glioblastoma (optionally secondary glioblastoma).

38. The pharmaceutical composition for use according to any one of claims 33-37, comprising an oral composition of zotiraciclib, or a pharmaceutically acceptable salt thereof.

39. The pharmaceutical composition for use according to any one of claims 33-35, comprising one or more additional agents, optionally selected from chemotherapeutic agents, hormonal therapeutic agents, and/or kinase inhibitors.

40. The pharmaceutical composition for use according to claim 39, wherein the chemotherapeutic agent comprises an IDH1 or IDH2 inhibitor, optionally ivosidenib (AG-120), enasidenib, or AG-221.

41. The pharmaceutical composition for use according to any one of claims 31-40, wherein the method comprises:
(a) determining IDH1 or IDH2 mutation status in a tissue sample from the subject; and
(b) administering to the subject the composition comprising zotiraciclib (TG02), or the pharmaceutically acceptable salt thereof, if the tissue sample comprises the IDH1 or IDH2 mutation.

42. Use of a composition in the preparation of a medicament for treating a cancer in a human subject in need thereof, wherein the cancer comprises an isocitrate dehydrogenase (IDH1 or IDH2) mutation, comprising zotiraciclib (TG02), or a pharmaceutically acceptable salt thereof.

43. The use according to claim 42, wherein the IDH1 or IDH2 mutation is a gain-of-function mutation characterized by increased conversion of α-ketoglutarate (α-KG) into an oncometabolite D-2-hydroxyglutarate (D-2HG) relative to homozygous wild-type IDH1 or IDH2.

44. The use according to claim 42 or 43, wherein the IHD1 mutation is R132X, wherein X is selected from any amino acid other than R, optionally wherein the IHD1 mutation is R132C, R132G, R132H, R132L, or R132S.

45. The use according to any one of claims 42-44, wherein the IHD2 mutation is R172X or R140X, wherein X is selected from any amino acid other R, optionally wherein the IHD2 mutation is R172G, R172K, R172M, R172S, R172T, or R140Q.

46. The use according to any one of claims 42-45, wherein the cancer is selected from glioma (optionally low-grade or high-grade glioma), medulloblastoma, chondrosarcoma, cholangiocarcinoma, acute myeloid leukemia (AML), astrocytoma, sinonasal undifferentiated carcinoma (SNUC), angioimmunoblastic T cell lymphoma (AITL), and glioblastoma (optionally secondary glioblastoma).

47. The use according to any one of claims 42-45, comprising an oral composition of zotiraciclib, or a pharmaceutically acceptable salt thereof.

48. The use according to any one of claims 42-47, comprising one or more additional agents, optionally selected from chemotherapeutic agents, hormonal therapeutic agents, and/or kinase inhibitors.

49. The use according to claim 49, wherein the chemotherapeutic agent comprises an IDH1 or IDH2 inhibitor, optionally ivosidenib (AG-120), enasidenib, or AG-221.

50. The use according to any one of claims 31-40, comprising:
(a) determining IDH1 or IDH2 mutation status in a tissue sample from the subject; and
(b) administering to the subject the composition comprising zotiraciclib (TG02), or the pharmaceutically acceptable salt thereof, if the tissue sample comprises the IDH1 or IDH2 mutation.
